(19) United States
(12) Patent Application Publication
(10) Pub. No.: US 2026/0015662 A1
(43) Pub. Date: Jan. 15, 2026

(54) DETECTING ANTIBIOTIC AND/OR CHEMICAL USE IN ANIMALS USING EPIGENETIC MEANS

(71) Applicant: Evonik Operations GmbH, Essen (DE)

(72) Inventors: Christos GIATSIS, Aschaffenburg (DE); Florian BÖHL, Neckargemünd (DE); Rose WHELAN, Ambrosden, Oxfordshire (GB); Stefan PELZER, Gütersloh (DE); Marie-Luise VOGEL, Bielefeld (DE); Frank LYKO, Hiedelberg (DE); Günter RADDATZ, Heidelberg (DE)

(73) Assignee: Evonik Operations GmbH, Essen (DE)

(21) Appl. No.: 18/881,923

(22) PCT Filed: Jul. 4, 2023
(86) PCT No.: PCT/EP2023/068426

§ 371 Date: Jan. 7, 2025

(30) Foreign Application Priority Data
Jul. 8, 2022 (EP) 22183740.4

(51) Int. Cl. C12Q 1/6876

(52) U.S. Cl. CPC C12Q 1/6876; C12Q 2600/142; C12Q 2600/154

(57) ABSTRACT

A method of determining if a terrestrial test animal and/or test animal from which a product is derived has been treated and/or is currently undergoing treatment with at least one antibiotic and/or veterinary chemical includes: (a) determining a test methylation profile from genomic material contained in a biological sample obtained from the test animal and/or the animal-derived product; and (b) comparing the test methylation profile obtained from (a) with a reference methylation profile obtained from a control animal of the same biological taxon of the test animal, where the control animal was not treated and/or is not currently undergoing treatment with at least one antibiotic and/or veterinary chemical, wherein a difference in the test methylation profile of (a) compared to the reference methylation profile from the control animal is indicative of the test animal having been treated and/or is currently undergoing treatment with at least one antibiotic and/or veterinary chemical.

### FIELD OF THE INVENTION

The present invention relates to a method for detecting the use and/or administration of at least one antibiotic and/or veterinary chemical to an animal using epigenetic means. In particular, the method is capable of detecting the use of at least one antibiotic or the administration of at least one antibiotic an/or veterinary chemical during the breeding or rearing of at least one terrestrial animal by determining the methylation status of a CpG site in a test animal and comparing the resultant methylation status with a reference methylation status of a control animal which was bred without the use of or administration of an antibiotic and/or veterinary chemical.

### BACKGROUND OF THE INVENTION

Antibiotics are used for the treatment of bacterial diseases in human and veterinary medicine. Antibiotics have also been used prophylactically at low levels in feed or water to improve growth rates and mortality levels in livestock. However, overuse of antibiotics especially in agriculture, has been associated with an increase in antibiotic resistant bacteria, which will eventually impair the ability to treat bacterial diseases in humans and animals alike. Additionally, antibiotic usage has been associated with other unintended and undesirable physiological consequences like low birth weight in infants from antibiotic treated mothers, increased disease susceptibility, and the like. Tools for assessing not only current, but historical antibiotic use would therefore be of high interest in livestock production for the purpose of guiding breeding, feeding, veterinary and/or management practices. Additionally, analysis of meat and meat products to assess historic antibiotic use in the individual animal prior to slaughter could be of high interest for auditing, certifying, and labelling of meat products.

To date antibiotic testing involves chemical assays to find residues of antibiotics present in the blood or tissues of animals. However, most antibiotics used for growth promotion are either added to the feed or water and most of these classes are poorly absorbed in the gastrointestinal system, so residue testing in blood or tissues may not capture usage of all antibiotic classes, particularly after the withdrawal period (where the animal is no longer given the antibiotics) commences. There is also currently no method by which animal or meat products can be tested for previous antibiotic usage unless antibiotic residues remain in the blood or tissues.

Accordingly, there is a need in the art for a simple and accurate means of detection of current and past antibiotic and/or drug use in animals, particularly livestock.

Epigenetics is the study of inherited traits caused by mechanisms other than changes in the underlying DNA sequence. In other words, epigenetic marks “orchestrate” our genes. Epigenetic marks can be either chemical (e.g., methylation), protein-based (e.g., histones) or a combination of the two. During development and cell differentiation, DNA methylation is dynamic, but some DNA methylation patterns may be retained as a form of epigenetic memory, accumulated and/or inherited to next generation. Those changes might be responsible for heritable changes in gene activity as DNA methylation events have been shown to be regulation mechanisms associated with gene silencing, expression, chromatin remodelling or imprinting. Epigenetics is attractive for animal breeding as it may identify causality and heritability of complex traits and diseases. DNA methylation patterns are modified along the life of an individual by environmental forces like diet, stress, drugs, or pollution among many others. Some environments are more likely to increase certain methylation patterns, and these patterns could contribute to the epigenetic and/or phenotypic variation between individuals.

Several studies have shown that antibiotic exposure can change epigenetic markers in eukaryotic cells. For example, in a study examining the relationship between antibiotic usage during pregnancy and birth weight outcomes, it was found that there were specific regions of the genome that were differentially methylated in response to antibiotic exposure (Vidal et al., 2013). A study conducted with cultured plant cells observed changes in global DNA methylation when the cells were exposed to the antibiotic kanamycin (Bardini et al., 2003). However, there is no mention in the art of epigenetic changes that may occur due to current and/or past antibiotic and/or drug use in animals, particularly livestock.

Accordingly, there is still a need in the art for a simple and accurate method for detection of current and/or past antibiotic and/or drug use in animals, particularly livestock.

### DESCRIPTION OF THE INVENTION

The present invention solves the problems above by providing a means of using alterations in DNA methylation patterns to develop novel analytics for assessment of antibiotic or other chemical usage in terrestrial agricultural animal production, meat and/or meat products. In particular, the means comprises a method of detecting the use of antibiotics and/or veterinary chemicals in a test animal and/or a test animal from which a product is derived by comparing the methylation status of at least one CpG site in the test animal and the corresponding CpG site in a control animal and/or a control animal from which a product is derived, where no antibiotics and/or veterinary chemicals were used on the control animal and, wherein the presence of hypomethylation or hypermethylation at the CpG site in the test animal is indicative of the test animal having been treated with antibiotics and/or veterinary chemicals. The method according to any aspect of the present invention is a simple and accurate method for detection of current and/or past antibiotic and/or drug use in animals.

The present invention is based on the finding that contact with antibiotics and/or veterinary chemicals to an animal, results in stress in the internal environment of the animal and this can permanently change the genome of the animal through epigenetics. In particular, the capability to adapt to the environment (i.e., presence of antibiotics and/or veterinary chemicals) and maintain the adapted biological pattern depends on epigenetic mechanisms, including DNA methylation. In particular, the present invention is based on the finding that contact with antibiotics and/or veterinary chemicals may also result in changes in epigenetic mechanisms of the animal, including DNA methylation patterns and these patterns may also be passed down to the different products that may derive from the animal.

The inventors have unexpectedly found that this property can be utilized to identify “epigenetic fingerprints” on the genome that are specific to the presence of antibiotics and/or veterinary chemicals of not just one animal but possibly all the animals that are brought into contact with at least one of antibiotic and/or veterinary chemical. For example, these ‘epigenetic fingerprints’ may be specific not only for the animal that was brought into contact with antibiotics and/or veterinary chemicals but also for any product that stems from the very animal (an individual). Based on these findings, the present invention provides means to detect use of antibiotics and/or veterinary chemicals in animals, in particular terrestrial animals, for example rearing animals also known as livestock and poultry from which an animal-derived product comes from. In this way, the method according to any aspect of the present invention may then be used to accurately and reliably determine if any test animal or product therefrom has been brought into contact or treated with at least one antibiotic and/or veterinary chemical. The method according to any aspect of the present invention may then use DNA methylation analysis to also differentiate usage of antibiotics prophylactically (growth promotion) from usage of antibiotics therapeutically. DNA methylation analysis may also be a step, in elucidating, whether an animal treated with veterinary chemicals and/or antibiotics was given a withdrawal period from the veterinary chemical and/or antibiotic prior to slaughter or differentiate usage of different classes of antibiotics. The method according to the present invention may also be used for assessment of current and historic antibiotic usage in livestock or meat to investigate epigenetic markers which can then subsequently be used for the creation of analytics to test antibiotic usage in terrestrial livestock species. Further, the method according to any aspect of the present invention may then be used to predict and avoid undesired traits contributed by use of antibiotics and/or veterinary chemicals to the animal welfare and to a more sustainable livestock production. In one example, the method according to any aspect of the present invention may also be used to differentiate the route of administration of antibiotics (i.e., water vs feed vs injection types).

According to one aspect of the present invention, there is provided a method of determining if a test animal and/or a test animal from which a product is derived has been treated and/or is currently undergoing treatment with at least one antibiotic and/or veterinary chemical, the method comprising:
(a) determining a test methylation profile from the genomic material contained in a biological sample obtained from the test animal and/or the animal-derived product; and
(b) comparing the test methylation profile obtained from (a) with a reference methylation profile obtained from a control animal, where the control animal was not treated and/or is not currently undergoing treatment with at least one antibiotic and/or veterinary chemical,
   wherein a difference in the test methylation profile of (a) compared to the reference methylation profile from the control animal, is indicative of the test animal having been treated and/or is currently undergoing treatment with at least one antibiotic and/or veterinary chemical; and
   wherein the test animal is a terrestrial animal and wherein the veterinary chemical is an anti-parasitic, an anti-viral, a feed additive, a water additive, a disinfectant, glutaraldehyde, and/or formalin.

In one example, the veterinary chemical is at least one anti-microbial such as an anti-parasitic, an anti-viral or antibiotic.

According to another aspect of the present invention, there is provided a method of determining if a test animal and/or a test animal from which a product is derived has been treated and/or is currently undergoing treatment with at least one antibiotic the method comprising:
(a) determining a test methylation profile from the genomic material contained in a biological sample obtained from the test animal and/or the animal-derived product; and
(b) comparing the test methylation profile obtained from (a) with a reference methylation profile obtained from a control animal, where the control animal was not treated and/or is not currently undergoing treatment with at least one antibiotic,
   wherein a difference in the test methylation profile of (a) compared to the reference methylation profile from the control animal, is indicative of the test animal having been treated and/or is currently undergoing treatment with at least one antibiotic.

According to a further aspect of the present invention, there is provided a method of determining if a test animal and/or a test animal from which a product is derived has been treated and/or is currently undergoing treatment with at least one antibiotic or veterinary chemical or a combination thereof, the method comprising:
(a) determining the methylation status of at least one CpG site in genomic material contained in a biological sample obtained from the test animal,
(b) comparing the methylation status of the CpG site from (a) with that of a control animal which was not treated and/or is not currently undergoing treatment with at least one antibiotic and/or veterinary chemical,
   wherein a difference in the test methylation status of (a) in the test animal compared to the CpG site in the control animal is indicative of the test animal having been treated and/or is currently undergoing treatment with at least one antibiotic and/or veterinary chemical; and
   wherein the test animal is a terrestrial animal and the veterinary chemical is an anti-parasitic, an anti-viral, a feed additive, a water additive, a disinfectant, glutaraldehyde, and/or formalin.

In one example, the veterinary chemical is at least one anti-microbial such as an anti-parasitic, an anti-viral or antibiotic.

The difference in methylation status according to any aspect of the present invention is hypomethylation or hypermethylation of the CpG site in the test animal and the hypomethylation or hypermethylation of the CpG site is indicative of the test animal having been treated and/or is currently undergoing treatment with at least one antibiotic and/or drug.

As used herein, the term “terrestrial animal” refers to any organism that lives entirely on land or that lives predominantly on land, especially compared with aquatic animals. In particular, the terrestrial animal according to any aspect of the present invention may be any animal in the animal kingdom. More in particular, the terrestrial animal according to any aspect of the present invention may be may a mammal or a bird. Even more in particular, the terrestrial animal according to any aspect of the present invention may be rearing animals selected from livestock or poultry. In particular, livestock may include cattle, sheep, pigs, goats, horses, camels, donkeys, mules, rabbits and the like and poultry may include chickens, turkeys and other gallinaceous birds, ducks, geese, quail, and the like. As used herein, the term ‘livestock’ may also include poultry and refer to any farm animal or animal that may be used in agriculture. More in particular, the terrestrial animal according to any aspect of the present invention may be selected from the group consisting of cow, sheep, pig, goat, horse, camel, donkey, mule, rabbit, chicken, turkey, duck, goose, and quail.

As used herein, the term ‘animal-derived product’ refers to products that originate from animals. In particular, the term ‘test animal-derived product’ refers to the sample or subject in question that is to undergo the method according to any aspect of the present invention. These products from animals may include meat and meat products, also including fat, flesh, blood, processed meat, and lesser-known products, such as isinglass and rennet, poultry products (meat and eggs), dairy products (milk and cheese), and non-food products such as fibre (wool, mohair, cashmere, leather, and the like). Animal-derived products may also include products that can be made using animal products (e.g., fat) such as soap, creams, and such. In one example, the animal-derived product is meat, eggs, blood, brain, sperm, milk and any other tissue or sample that provides genomic DNA. In particular, the animal-derived product is meat. In one example, the animal-derived product sample may be a single type of meat, different types of meat, a single part of a type of meat, different parts of a single type of meat or different parts of different types of meat. The sample may be from any biological entity having a DNA genome and DNA genome methylation. In particular, the methylation site is a CpG site.

In one example, the test animal and/or test animal derived product may be selected from the group consisting of pig for fattening, cattle for fattening, piglet, turkey for fattening, chicken for fattening, veal calf, horse, sheep meat, rabbit for fattening, sheep dairy products, horse for fattening, lamb for fattening, sheep for fattening, cow dairy products, laying hen, sow with piglets, goat dairy products and the like.

The term “test” used in conjunction with the term subject and/or animal in the present disclosure refers to an entity that is subjected to the method according to any aspect of the present invention and is the basis for an analysis application of the present invention. An “(individual) test subject”, an “(individual) group of test subjects” or a “test profile” or an ‘test animal derived product’ is therefore a (individual) subject or group of subjects being tested according to the invention or a profile being obtained or generated in this context. Conversely, the term “reference” or ‘control’ shall denote, mostly predetermined, entities which are used for a comparison with the test entity. For example, the term ‘reference animal’ used interchangeably with ‘control animal’ refers to an animal of the same biological taxon used for comparison or as a control in reference to the ‘test animal’. Similarly, the term ‘sample’ and/or ‘test animal-derived product sample’ used in accordance with any aspect of the present invention refers to an entity that may be subject to the method of the present invention. In particular, a sample may be any (test) animal-derived product that may be subject to the method of the present invention to determine if the test animal has had or has contact with at least one antibiotic and/or veterinary chemical from which the test animal-derived product is obtained from. For example, an animal-derived product sample may be a piece of meat tested according to any aspect of the present invention to determine if the test animal has had contact with at least one antibiotic and/or veterinary chemical from which the test animal-derived product is obtained from. In another example, a (test) animal-derived product sample may be milk which is tested according to any aspect of the present invention to determine if the test animal currently still has contact with at least one antibiotic and/or veterinary chemical from which the test animal-derived product is obtained from. Blockchain may also be used to make the information easily available for the consumer.

As used herein, the term “comprising” is to be construed as encompassing both “including” and “consisting of”, both meanings being specifically intended, and hence individually disclosed aspects of the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g., “a”, “an” or “the”, this includes a plural of that noun unless something else is specifically stated.

In context of the present invention, the terms “methylation profile”, “methylation pattern”, “methylation state” or “methylation status,” are used herein to describe the state, situation, or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The DNA segment may also include specific pre-selected methylation sites.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “methylation status” refers to the status of a specific methylation site (i.e., methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an animal derived product or animal that has not had contact with antibiotics and/or veterinary chemicals.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an animal derived product or animal that has not had contact with antibiotics and/or veterinary chemicals.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine, and guanine. Typical bases for RNA are uracil, adenine, cytosine, and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in an animal that was brought into contact with an antibiotic and/or veterinary chemical compared to a cell with no contact with an antibiotic and/or veterinary chemical.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745). In context of the present invention, the terms “methylation profile”, “methylation pattern”, “methylation state” or “methylation status,” are used herein to describe the state, situation, or condition of methylation of a genomic sequence, and such terms refer to the characteristics of a DNA segment at a particular genomic locus in relation to methylation.

Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

As used herein, a “methylated nucleic acid molecule” refers to a nucleic acid molecule that contains one or more nucleotides that is/are methylated.

The term ‘epigenetic change’ as used herein refers to a chemical (e.g., methylation) change or protein (e.g., histones) change that takes place to a gene body or a promoter thereof. Through epigenetic changes, environmental factors like. diet, stress, and prenatal nutrition can make an imprint on genes passed from one generation to the next.

As used herein, the term “genomic material” refers to nucleic acid molecules or fragments of the genome of the subject or group of subjects. In particular, such nucleic acid molecules or fragments are DNA or RNA or hybrids thereof, and most preferably are molecules of the DNA genome of a subject or group of subjects.

As used herein, the “DNA sample” refers to the DNA extracted from the terrestrial animal according to any aspect of the present invention using known methods in the art.

In particular, when there is differential methylation detected in a test animal, that is to say that the test animal and/or a product derived from the test animal displays hypermethylation or hypomethylation at, at least one CpG site in comparison to the control (i.e., an animal without contact with at least one antibiotic and/or veterinary chemical), then the test animal has or has had contact with at least one antibiotic and/or veterinary chemical. The difference in methylation according to any aspect of the present invention may be hypomethylation or hypermethylation.

A “biological sample” in context of the invention may comprise any biological material obtained from the subject or group of subjects that contains genomic material, and may be liquid, solid or both, may be tissue or bone, or a body fluid such as blood, lymph, etc. In particular, the biological sample useful for the present invention may comprise biological cells or fragments thereof. More in particular, the biological sample is selected from the group consisting of blood, brain, sperm, milk and any other tissue or sample that provides genomic DNA.

The one or more pre-selected methylation sites in (a) are methylation sites associated with tissue specific gene expression, preferably wherein the pre-selected methylation sites are associated with gene expression of one distinct tissue.

The tissue may be selected from
(i) metabolic tissue such as gut tissue, said gut tissue preferably being ileum or jejunum,
(ii) muscular tissue,
(iii) skin tissue, and
(iv) organ tissue, said organ tissue preferably being hepatic and/or pancreatic tissue.

As used herein the term ‘antibiotic’ refers to any medicine that may be fed to the terrestrial animal for therapeutic and/or preventive purposes. The antibiotic may be administered by any method known in the art. The antibiotic may be fed orally to the terrestrial animal according to any aspect of the present invention in the animal feed, or water such that it is ingested. In another example, the antibiotic may be injected into the animal. In one example, the antibiotic may be introduced into the terrestrial animal via udder injections. A skilled person would understand the best way to provide the antibiotic to the animal based on the specific biological taxon of the animal, the type of antibiotic and the disease to be treated or prevented. In particular, the antibiotic according to any aspect of the present invention may be selected from the group of classes consisting of amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim. More in particular, the antibiotic may be selected from the group consisting of tetracycline hydrochloride, Amoxicillin and Colist.

The test animal according to any aspect of the present invention may be fed with at least one or more antibiotics mentioned above simultaneously or consecutively. The contact of antibiotics with the terrestrial animal may bring about epigenetic changes, at least DNA methylation changes, that may then be determined using the method according to any aspect of the present invention. The concentration of antibiotics in each dose and/or the period of time the antibiotic has been given to the test animal may affect the extend of differential methylation in the test animal relative to the control animal. It is within the knowledge of a skilled person to determine the concentration of each dose and the period of antibiotic exposure that the test animal requires depending on whether the antibiotic is given for preventive or therapeutic measures.

As used herein the term ‘veterinary chemical’ refers to drugs or medicines used to treat or prevent disease, injury, and pests in animals. In particular, ‘veterinary chemical’ may refer to an anti-parasitic, an anti-viral, a feed additive, a water additive, a disinfectant, glutaraldehyde, formalin, mixtures thereof and the like. In one example, the feed additive may be a coccidiostat or ionophore. Water additive refers to chemicals that may be added to water lines rather than into feed of the terrestrial animals. The veterinary chemical may be administered by any method known in the art to the terrestrial animal. In particular, the veterinary chemical refers to at least one anti-microbial such as an anti-parasitic, an anti-viral or antibiotic.

The test animal used in the method according to any aspect of the present invention may be brought into contact with both an antibiotic and a veterinary chemical simultaneously and/or consequently. The change in the internal environment of the test animal leads to an epigenetic change and this can be determined using the method according to any aspect of the present invention.

In particular, in the method according to any aspect of the present invention, in step (a) the methylation status of at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 CpG sites are determined. A skilled person would be capable of determining the number of CpG sites that need to be used in step (a) according to any aspect of the present invention. Even more in particular, the methylation status of at least two CpG sites are determined in step (a) of the method according to any aspect of the present invention.

The method according to any aspect of the present invention, further comprises the step of:
(i) performing bisulfite modification to the DNA sample before step (a).

‘Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the present invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

Accordingly, before step (a) according to any aspect of the present invention is carried out, the genomic DNA contained/obtained or extracted from the cell, is first bisulfite treated.

An alternative method available in the art may be used instead of bisulfite treatment. A skilled person will understand which other methods to use. In one example, TET-assisted pyridine borane sequencing (TAPS) may be used for detection of 5mC and 5hmC (Yibin Liu, et al., Nature Biotechnology, 37: 424-429 (2019).

The cell used according to any aspect of the present invention is obtained from a biological sample selected from the group consisting of blood, brain, sperm and any other tissue or sample that provides genomic DNA to be used in the method according to any aspect of the present invention. In particular, the biological sample may comprise any biological material obtained from the subject that contains DNA, and may be liquid, solid or both, may be tissue or bone, or a body fluid such as blood, lymph, etc. In particular, the biological sample useful for the present invention may comprise biological cells or fragments thereof.

According to a further aspect of the present invention, there is provided a method of determining if a test animal and/or a test animal from which a product is derived has been treated and/or is currently undergoing treatment with at least one antibiotic, and if so, determining the distinct class of antibiotics with which the test animal is being treated and/or is currently undergoing treatment, the method comprising:
(a) determining a test methylation profile from the genomic material contained in a biological sample obtained from the test animal and/or the animal-derived product sample; and
(b) comparing the test methylation profile obtained from (a) with one or more predetermined reference methylation profiles, wherein each of the predetermined reference methylation profiles is from a different animal of the same biological taxon as the test animal, and each of the different animals was treated with a different class of antibiotics,
   wherein if the test methylation profile of (a) is significantly similar to one of the predetermined reference methylation profiles, the test animal is confirmed for being treated with the same class of antibiotics with which the animal with the predetermined reference profile is treated with and the test animal has been confirmed for having been treated and/or is currently undergoing treatment with the distinct class of antibiotics; and wherein the test animal is a terrestrial animal.

The distinct classes of antibiotics are amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim.

In one example, a panel of pre-determined reference profiles may be prepared for different animals to be used as a control where each animal has been contacted with a different class of antibiotics and/or each part of the animal (i.e., tissue, muscle, blood, skin) has its own unique pre-determined methylation reference profile that also forms a part of the panel of pre-determined reference profiles. Different animals from the same biological taxon as the test animal, each being treated with a different class of antibiotic may have its own panel of pre-determined reference profiles for each part of the animal or animal-derived product that is used as the genomic material. For example, each panel may be specific for a single animal in contact with a first antibiotic and/or veterinary chemical where each reference profile may be distinct for a part of the animal from which the genomic material is extracted. There will thus be a compilation of panels of pre-determined reference profiles, each panel specific for one control animal of the same biological taxon as the test animal, the control animal being in contact with or is in contact with a first, second, third and the like antibiotic and/or veterinary chemical. When a test methylation profile from an unknown animal-derived product sample is obtained, this is then compared with the different panels of pre-determined reference profiles for the same animal taxon as the test animal to determine the distinct class of antibiotic and/or veterinary chemical the test animal is or was in contact with.

According to yet another aspect of the present invention refers to a method of determining if a test animal and/or a test animal from which a product is derived has been treated and/or is currently undergoing treatment with at least one antibiotic, and if so, determining if the antibiotic is used as a growth promotant or as a therapeutant, the method comprising:
(a) determining a test methylation profile from the genomic material contained in a biological sample obtained from the test animal and/or the animal-derived product; and
(b) comparing the test methylation profile obtained from (a) with one or more predetermined reference methylation profiles, wherein each of the predetermined reference methylation profiles is from a different control animal of the same biological taxon as the test animal, and each of the different control animals was treated with the antibiotic as a growth promotant or therapeutant,
   wherein if the test methylation profile of (a) is significantly similar to one of the predetermined reference methylation profiles, the test animal is confirmed for being treated with the antibiotic in the same way that the control animal with the similar predetermined reference profile is treated and the test animal has been confirmed for having been treated and/or is currently undergoing treatment with the antibiotic as a growth promotant or therapeutant; and wherein the test animal is a terrestrial animal.

As used herein the term ‘growth promotant’ refers to the antibiotic being used to help increase the efficiency of animal production by increasing weight gain and product output. The antibiotic may be used as a growth promotant in contrast to it being used as a therapeutant (i.e., for treatment of a disease) According to a further aspect of the present invention, there is provided a method of determining if a test animal from which a product is derived underwent a withdrawal period of no treatment with at least one antibiotic and/or veterinary chemical prior to the product being obtained, the method comprising:
(a) determining a test methylation profile from the genomic material contained in a biological sample obtained from the animal-derived product; and
(b) comparing the test methylation profile obtained from (a) with at least two predetermined reference methylation profiles, wherein at least one of the predetermined reference methylation profiles is from a control animal that underwent a withdrawal period and at the other one of the predetermined reference methylation profiles is from a control animal of the same biological taxon as the test animal, that did not undergo a withdrawal period before the product was obtained,
   wherein if the test methylation profile of (a) is significantly similar to the predetermined reference methylation profile, the test animal is confirmed for having undergone a withdrawal period or not; and
   wherein the test animal is a terrestrial animal.

As used herein, the term ‘withdrawal period’ refers to the period from the time point where the animal is no longer fed the antibiotic and/or veterinary chemical to the point where the remaining antibiotic is broken down in the body until it becomes a non-functional agent and is finally, eliminated from the body of the animal. Withdrawal periods of different antibiotics may vary from 1 or 2 days to couple of weeks. A “withdrawal” period is required from the time antibiotics are administered until it is legal to slaughter the animal or to derive products from the animal. The time it therefore takes the body to break down the antibiotic until it is no longer functional, or present is called the withdrawal time (or withdrawal period). Once the withdrawal period has passed the antibiotic has been eliminated from the animal's system.

According to another aspect of the present invention, there is provided a use of DNA methylation profiling for determining if a test animal and/or a test animal from which a product is derived has been treated and/or is currently undergoing treatment with at least one antibiotic and/or veterinary chemical, wherein the test animal is a terrestrial animal and the veterinary chemical is an anti-parasitic, an anti-viral, a feed additive, a water additive, a disinfectant, glutaraldehyde, and/or formalin.

In one example, the veterinary chemical is at least one anti-microbial such as an anti-parasitic, an anti-viral or antibiotic.

According to any aspect of the present invention, test methylation profile and reference methylation profile may be obtained by determining the methylation status of a pre-selected group of CpG sites in the genome of the test and reference animal respectively. The CpG site according to any aspect of the present invention may be selected from the Table A below. In particular, the CpG site according to any aspect of the present invention is at least one CpG site selected from the Table A below:

TABLE A
CpG sites of a test or reference animal that may
be differentially methylated in the animal exposed
to antibiotic and/or veterinary chemical.

| No. | Chromosome | Position |
|---|---|---|
| 1 | 20 | 10783159 |
| 2 | 24 | 4184075 |
| 3 | 25 | 1910205 |
| 4 | 4 | 1712424 |
| 5 | 4 | 50270218 |
| 6 | 5 | 57738909 |
| 7 | 7 | 10956706 |
| 8 | 12 | 18699814 |
| 9 | 12 | 2415548 |
| 10 | 17 | 4082979 |
| 11 | 18 | 9932606 |
| 12 | 2 | 29649123 |
| 13 | 23 | 4069359 |
| 14 | 25 | 1445548 |
| 15 | 28 | 3467160 |
| 16 | 3 | 10490126 |
| 17 | 3 | 3328494 |
| 18 | 4 | 1627649 |
| 19 | 6 | 11522056 |
| 20 | 6 | 23106717 |
| 21 | 8 | 5481923 |
| 22 | 33 | 716102 |
| 23 | 22 | 4604122 |
| 24 | 22 | 4617202 |
| 25 | 23 | 502108 |
| 26 | 24 | 5683979 |
| 27 | 27 | 3015432 |
| 28 | 3 | 10479779 |
| 29 | 19 | 5380287 |
| 30 | 15 | 12638203 |
| 31 | 10 | 20073849 |
| 32 | 5 | 55805858 |

### EXAMPLES

The foregoing describes preferred embodiments, which, as will be understood by those skilled in the art, may be subject to variations or modifications in design, construction, or operation without departing from the scope of the claims. These variations, for instance, are intended to be covered by the scope of the claims.

### Example 1

An in vitro to investigate the effects of three different antibiotics on the DNA methylation quantity in a swine intestinal cell line.

An immortalised cell line from swine intestines (IPEC2-J2 ACC 701 from DSMZ cell culture) was utilized in the 7^{th} passage. In a 12 well, flat-bottom plate, 1×10⁴ cel|s/well were seeded with DPBS D8537 (Sigma-Aldrich) and initially incubated for 4 days. After incubation cells were treated with one of 4 treatments for up to an additional 4 days:
1) Incubation for 4 days without any antibiotics
2) Incubation for 1 day with antibiotics followed by incubation for 1-3 days in antibiotic free media
3) Incubation for 2 days with antibiotics followed by incubation for 1-2 days in antibiotic free media
4) Incubation for 3 days with antibiotics followed by incubation for 1 day in antibiotic free media

Treatments 2, 3 and 4 were repeated with 3 different antibiotic treatments; tetracycline hydrochloride at 200 mg/L, Amoxicillin at 100 mg/L and Colistin at 480 mg/L. Duplicate cells per treatment were harvested at each time point. Media was removed, cells were washed once with DPBS, and a cell scraper was used to disassociate the cells from the bottom of the well. Cell pellets were then stored at −80° C. until DNA could be isolated using the NuceloSpin® DNA Stool Kit (Macherey and Nagel). Methylation grade of 100ng of isolated DNA was determined using the Imprint® Methylated DNA Quantification Kit (Sigma-Aldrich) which compared samples to a standard curve of known methylated DNA.

TABLE 1
The average amount of methylated DNA (ng/100 ng isolated DNA) isolated from IPEC2 cells
incubated in media without antibiotics or with either 200 mg/L Tetracycline Hydrochloride
(Tet), 100 mg/L Amoxicillin (Amx) or 480 mg/L Colistin (Col) over 4 days.

| Treatment | Day 0 | Day 1 | Day 2 | Day 3 | Day 4 |
|---|---|---|---|---|---|
| 4 Days without antibiotics | 27.8 ± 2.4¹ | 27.7 ± 19.7 | 35.0 ± 11.6 | 16.1 ± 2.6 | 31.6 ± 1.4 |
| 1 Day Tet before washing | NA² | −5.6 ± 2.9 | 12.0 ± 6.9 | 18.2 ± 16.0 | 25.8 ± 22.4 |
| 2 Day Tet before washing | | | 0.1 ± 8.1 | −8.6 ± 11.1 | 7.4 ± 9.7 |
| 3 Day Tet before washing | | | | 6.0 ± 13.2 | 21.7³ |
| 1 Day Amx before washing | | 4.1 ± 8.9 | 12.3 ± 3.2 | 20.4 ± 8.3 | 19.0 ± 2.1 |
| 2 Day Amx before washing | | | 20.9 ± 10.4 | 9.8 ± 4.7 | 21.3 ± 3.4 |
| 3 Day Amx before washing | | | | 26.0 ± 10.2 | 10.2 ± 9.4 |
| 1 Day Col before washing | | 26.1 ± 5.0 | 11.7 ± 6.8 | 19.6 ± 5.5 | 24.9 ± 0.8 |
| 2 Day Col before washing | | | 29.5 ± 3.8 | 1.4 ± 7.9 | 19.0 ± 5.4 |
| 3 Day Col before washing | | | | 29.1 ± 18.0 | 28.5 ± 16.4 |

¹Represents the standard error of the mean for the reported average
²NA, Not Applicable
³Standard error of the mean could not be calculated as one of the two replicates had insufficient isolated DNA

The results indicate that all three antibiotics resulted in a reduction in overall DNA methylation within 24 hrs of treatment. This effect was largest in Tetracycline, followed by Amoxicillin and finally Colistin treated cells when compared with the antibiotic free control cells. The differential methylation was also partially reverted within 1-3 after cells were washed and antibiotic treated media was replaced with antibiotic-free media. The results signify that antibiotic treatment can alter the methylation status of eukaryotic cells, and that these effects may be specific to antibiotic class and duration of treatment.

### Example 2

Impact of three classes of antibiotics administered as either growth promoters or therapeutically (either with or without withdrawal period) on the methylome of broiler chickens.

A study was conducted to determine whether methylation of CpG sites in the genome of chicken breast tissue and blood could be differentially affected by:
1) The administration of an antibiotic for growth promotion
2) The administration of antibiotics as a therapeutic
3) The administration of localized antibiotic classes
4) The administration of systemically absorbed antibiotic classes
5) The administration of therapeutic doses of antibiotics with or without a withdrawal period prior to slaughter

To create the treatments for the in vivo chicken trial, three different antibiotics were utilized (Table 2).

TABLE 2
Antibiotic classes, modes of action, absorption level and withdrawal period for the 3

| | | | Recommended |
|---|---|---|---|
| Antibiotic | | | Withdrawal for |
| (Class) | Antibiotic Mode of Action | Absorption | Broilers |
| Bacitracin Methylene | Inhibits peptidoglycan synthesis | Local | None required |
| Disalicylate (Peptide) | Used as an AGP or therapeutically | | |
| Tylosin (Macrolide) | Inhibits protein synthesis | Systemic | 5 days |
| | Used therapeutically | | |
| Amoxicillin (Beta | Inhibits peptidoglycan synthesis | Systemic | 1-4 days |
| Lactam) | Used therapeutically | | |

antibiotics utilized to create treatments in an in vivo chicken trial.

A chicken trial was conducted from day of hatch to 42 days to represent the average slaughter age of broiler chickens in Europe. A total of 870 Ross308 male broilers were separated into 58 floor pens containing 15 birds/pen. Corn-soybean meal-based broiler diets were formulated to meet all nutrient requirements for starter (day 0-14), grower (day 14-28) and finisher (day 28-42) feeding phases. Treatments were created by adding different quantities of antibiotics to the basal diet for different durations (Table 3).

TABLE 3
Description of control and antibiotic fed treatments
in a 42-day broiler chicken trial.

| Treatments | Pen Replicates | Antibiotic Dose, kg/MT feed |
|---|---|---|
| 1. Negative control (no antibiotic) | 10 | — |
| 2. BMD - AGP Dose | 8 | 50 g/MT feed day 1-42 |
| 3. BMD - Therapeutic Dose | 8 | 200 g/MT Feed Day 23-28 |
| 4. Tylosin - withdrawal | 8 | 2 kg/MT Feed Day 32-37 |
| 5. Tylosin - no withdrawal | 8 | 2 kg/MT Feed Day 32-42 |
| 6. Amoxicillin - withdrawal | 8 | 1.86 kg/MT Feed Day 32-37 |
| 7. Amoxicillin - no withdrawal | 8 | 1.86 kg/MT Feed Day 37-42 |

Two animals per pen were the sacrificed for sampling on days 28 (for treatments 1-3) and 42 (all treatments) for extraction of blood and various tissues including breast muscle tissue (Pectoralis major). A total of 96 samples were then selected for DNA isolation with Invitrogen™ Purelink™ Genomic DNA Minikit (ThermoFisher Scientific, Waltham, MA, USA) and subsequent reduced bisulphite sequencing to compare methylation level of CpG sites in the genome:

$$Day 28 breast and blood from 4 birds / treatment = ( 4 birds \times 3 treatments ) \times ( 2 tissues / bird ) = 24 samples$$

$$Day 42 breast and blood from 5 birds / treatment = ( 5 birds \times 7 treatments ) \times ( 2 tissues / bird ) = 70 samples$$

$$Day 42 additional blood sample from Treatment 1 and Treatment 3 = 2 samples$$

$$Total = 96 samples$$

The resulting libraries were sequenced and mapped to the Gallus gallus 5.0 genome using the tool bsmap 2.9.

From the resulting methylation ratios all CpGs were kept, which had a non-zero coverage in all individual samples for a certain tissue. This resulted in 1,078,713 CpGs for breast and 1,077,240 CpGs for blood.

To further analyze the ability to classify the samples based on the methylation data for treatments with different antibiotics, the R package randomForest was used and a random forest-based classification was set up, using the 100,000 most variable CpGs in each tissue.

First, the set of CpGs was split in chunks of 10,000 and each chunk was used to classify the samples into different categories, using a random forest. This was repeated 100 times. All CpGs having a positive score “MeanDecreaseAccuracy” in an individual classification were pooled and the top 10,000 CpGs, occurring most often, were kept. Then a second random forest was set up, using these selected 10,000 CpGs as features and a classification was performed. This was repeated 100 times and the average classification error, as well as the optimal classification error was evaluated.

The results showed no difference in growth (body weight gain, feed intake or feed conversion ratio) of broilers in the different antibiotic treated groups. This is likely due to the very clean conditions and optimal nutrition these birds received during the trial, as antibiotics were then not needed to improve growth performance or prevent illness. Therefore, bird weight or health status did not need to be corrected for when comparing the treatments for methylation of CpG sites.

The results of the random forest analysis of CpG methylation identified that the tylosin withdrawal and amoxicillin treatment groups were distinguishable from the control with error rates of <0.05 when considering the best classification (Table 4). The BMD treatments and the Tylosin treatment without a withdrawal period were also distinguishable from the control group, although the best classification error rates were slightly higher between 0.056-0.111 in these treatments.

TABLE 4
Random forest analysis of CpG methylation based on the 100,000
most variable CpGs in breast tissue genomic DNA samples from
antibiotic treated broiler chickens compared to controls.

| | | Average | Best | Number of |
|---|---|---|---|---|
| | | Classification | Classification | Features in Best |
| Group 1 | Group 2 | Error | Error | Classification |
| 1. Control | 2. BMD - AGP Dose | 0.314 | 0.111 | 38 |
| 1. Control | 3. BMD - Therapeutic Dose | 0.275 | 0.056 | 34 |
| 1. Control | 4. Tylosin - Withdrawal | 0.169 | 0.000 | 49 |
| 1. Control | 5. Tylosin - No Withdrawal | 0.189 | 0.071 | 26 |
| 1. Control | 6. Amoxicillin - Withdrawal | 0.161 | 0.000 | 41 |
| 1. Control | 7. Amoxicillin - No Withdrawal | 0.165 | 0.000 | 35 |

When random forest analysis was conducted on the 100,000 most variable CpGs in blood samples from the different treatments, the best classification error rates were under 0.100 for the differentiation of each antibiotic treatment from the control group (Table 5).

The location of differentially methylated cytosine residues in antibiotic treated compared to control chickens are listed for breast (Table 6) and blood (Table 7).

These results indicate that treatment of broiler chickens with antibiotics results in significant shifts to the CpG methylation profiles, which may therefore be used to differentiate antibiotic-free from antibiotic-treated poultry meat regardless of the antibiotic class, purpose or usage of a withdrawal period.

TABLE 5
Random forest analysis of CpG methylation based on the 100,000 most variable CpGs in blood
genomic DNA samples from antibiotic treated broiler chickens compared to controls.

| | | Average | Best | Number of |
|---|---|---|---|---|
| | | Classification | Classification | Features in Best |
| Group 1 | Group 2 | Error | Error | Classification |
| 1. Control | 2. BMD - AGP Dose | 0.245 | 0.000 | 42 |
| 1. Control | 3. BMD - Therapeutic Dose | 0.256 | 0.062 | 38 |
| 1. Control | 4. Tylosin - Withdrawal | 0.184 | 0.000 | 29 |
| 1. Control | 5. Tylosin - No Withdrawal | 0.205 | 0.071 | 32 |
| 1. Control | 6. Amoxicillin - Withdrawal | 0.147 | 0.000 | 33 |
| 1. Control | 7. Amoxicillin - No Withdrawal | 0.194 | 0.000 | 34 |

TABLE 6
Genome sites of the CpG cytosine residues differentially methylated in blood extracted
DNA from the control group compared to each individual antibiotic treatment group.

| | Chromosome_{—} | | Chromosome_{—} | | Chromosome_{—} |
|---|---|---|---|---|---|
| Internal ID | Methylated C | Internal ID | Methylated C | Internal ID | Methylated C |
| Abx_Brs_1v3_02 | chr1_ [symbol] | Abx_Brs_1v6_17 | chr20_ [symbol] | Abx_Brs_1v3_20 | chr3_ [symbol] |
| Abx_Brs_1v5_01 | chr1_ [symbol] | Abx_Brs_1v5_13 | chr20_ [symbol] | Abx_Brs_1v4_34 | chr3_ [symbol] |
| Abx_Brs_1v5_02 | chr1_ [symbol] | Abx_Brs_1v4_16 | chr20_ [symbol] | Abx_Brs_1v3_21 | chr3_ [symbol] |
| Abx_Brs_1v7_01 | chr1_ [symbol] | Abx_Brs_1v2_17 | chr20_ [symbol] | Abx_Brs_1v5_18 | chr3_ [symbol] |
| Abx_Brs_1v2_01 | chr1_ [symbol] | Abx_Brs_1v6_18 | chr20_ [symbol] | Abx_Brs_1v [symbol] _33 | chr3_ [symbol] |
| Abx_Brs_1v3_01 | chr1_ [symbol] | Abx_Brs_1v3_09 | chr20_ [symbol] | Abx_Brs_1v3_22 | chr3_ [symbol] |
| Abx_Brs_1v4_01 | chr1_ [symbol] | Abx_Brs_1v4_17 | chr20_ [symbol] | Abx_Brs_1v4_30 | chr3_ [symbol] |
| Abx_Brs_1v6_01 | chr10_ [symbol] | Abx_Brs_1v3_10 | chr20_ [symbol] | Abx_Brs_1v2_30 | chr3_ [symbol] |
| Abx_Brs_1v7_02 | chr10_ [symbol] | Abx_Brs_1v2_14 | chr20_ [symbol] | Abx_Brs_1v7_27 | chr3_ [symbol] |
| Abx_Brs_1v [symbol] _03 | chr10_ [symbol] | Abx_Brs_1v2_13 | chr20_ [symbol] | Abx_Brs_1v6_32 | chr3_ [symbol] |
| Abx_Brs_1v [symbol] _03 | chr10_ [symbol] | Abx_Brs_1v2_15 | chr20_ [symbol] | Abx_Brs_1v7_28 | chr3_ [symbol] |
| Abx_Brs_1v6_04 | chr10_ [symbol] | Abx_Brs_1v4_14 | chr20_ [symbol] | Abx_Brs_1v4_28 | chr3_ [symbol] |
| Abx_Brs_1v5_04 | chr10_ [symbol] | Abx_Brs_1v4_15 | chr20_ [symbol] | Abx_Brs_1v4_31 | chr3_ [symbol] |
| Abx_Brs_1v6_02 | chr10_ [symbol] | Abx_Brs_1v2_16 | chr20_ [symbol] | Abx_Brs_1v4_32 | chr3_ [symbol] |
| Abx_Brs_1v2_02 | chr10_ [symbol] | Abx_Brs_1v2_18 | chr21_ [symbol] | Abx_Brs_1v4_33 | chr3_ [symbol] |
| Abx_Brs_1v4_02 | chr10_ [symbol] | Abx_Brs_1v6_19 | chr21_ [symbol] | Abx_Brs_1v3_25 | chr33_ [symbol] |
| Abx_Brs_1v7_03 | chr12_ [symbol] | Abx_Brs_1v6_20 | chr21_ [symbol] | Abx_Brs_1v3_ [symbol] | chr33_ [symbol] |
| Abx_Brs_1v3_04 | chr12_ [symbol] | Abx_Brs_1v7_15 | chr21_ [symbol] | Abx_Brs_1v3_23 | chr33_ [symbol] |
| Abx_Brs_1v3_05 | chr12_ [symbol] | Abx_Brs_1v3_11 | chr21_ [symbol] | Abx_Brs_1v3_24 | chr33_ [symbol] |
| Abx_Brs_1v6_07 | chr12_ [symbol] | Abx_Brs_1v2_19 | chr21_ [symbol] | Abx_Brs_1v [symbol] _34 | chr33_ [symbol] |
| Abx_Brs_1v4_06 | chr12_ [symbol] | Abx_Brs_1v4_18 | chr22_ [symbol] | Abx_Brs_1v2_31 | chr4_ [symbol] |
| Abx_Brs_1v4_03 | chr12_ [symbol] | Abx_Brs_1v4_19 | chr22_ [symbol] | Abx_Brs_1v7_29 | chr4_ [symbol] |
| Abx_Brs_1v [symbol] _03 | chr12_ [symbol] | Abx_Brs_1v7_17 | chr22_ [symbol] | Abx_Brs_1v5_19 | chr4_ [symbol] |
| Abx_Brs_1v5_05 | chr12_ [symbol] | Abx_Brs_1v4_20 | chr22_ [symbol] | Abx_Brs_1v6_35 | chr4_ [symbol] |
| Abx_Brs_1v4_04 | chr12_ [symbol] | Abx_Brs_1v7_ [symbol] | chr22_ [symbol] | Abx_Brs_1v6_36 | chr4_ [symbol] |
| Abx_Brs_1v4_05 | chr12_ [symbol] | Abx_Brs_1v3_12 | chr23_ [symbol] | Abx_Brs_1v7_30 | chr4_ [symbol] |
| Abx_Brs_1v6_05 | chr12_ [symbol] | Abx_Brs_1v5_14 | chr23_ [symbol] | Abx_Brs_1v5_20 | chr4_ [symbol] |
| Abx_Brs_1v6_0 [symbol] | chr12_ [symbol] | Abx_Brs_1v3_13 | chr23_ [symbol] | Abx_Brs_1v2_32 | chr4_ [symbol] |
| Abx_Brs_1v5_0 [symbol] | chr12_ [symbol] | Abx_Brs_1v7_18 | chr23_ [symbol] | Abx_Brs_1v4_36 | chr4_ [symbol] |
| Abx_Brs_1v5_08 | chr13_ [symbol] | Abx_Brs_1v6_21 | chr23_ [symbol] | Abx_Brs_1v4_35 | chr4_ [symbol] |
| Abx_Brs_1v6_08 | chr13_ [symbol] | Abx_Brs_1v3_14 | chr23_ [symbol] | Abx_Brs_1v3_27 | chr4_ [symbol] |
| Abx_Brs_1v6_09 | chr13_ [symbol] | Abx_Brs_1v4_21 | chr24_ [symbol] | Abx_Brs_1v3_28 | chr4_ [symbol] |
| Abx_Brs_1v6_ [symbol] | chr13_ [symbol] | Abx_Brs_1v7_19 | chr24_ [symbol] | Abx_Brs_1v4_38 | chr5_ [symbol] |
| Abx_Brs_1v7_ [symbol] | chr13_ [symbol] | Abx_Brs_1v6_22 | chr24_ [symbol] | Abx_Brs_1v4_39 | chr5_ [symbol] |
| Abx_Brs_1v7_07 | chr13_ [symbol] | Abx_Brs_1v3_16 | chr24_ [symbol] | Abx_Brs_1v3_31 | chr5_ [symbol] |
| Abx_Brs_1v8_11 | chr13_ [symbol] | Abx_Brs_1v2_20 | chr24_ [symbol] | Abx_Brs_1v2_34 | chr5_ [symbol] |
| Abx_Brs_1v [symbol] _03 | chr13_ [symbol] | Abx_Brs_1v5_15 | chr24_ [symbol] | Abx_Brs_1v4_37 | chr5_ [symbol] |
| Abx_Brs_1v3_06 | chr13_ [symbol] | Abx_Brs_1v6_ [symbol] | chr24_ [symbol] | Abx_Brs_1v5_21 | chr5_ [symbol] |
| Abx_Brs_1v7_04 | chr13_ [symbol] | Abx_Brs_1v5_ [symbol] | chr24_ [symbol] | Abx_Brs_1v3_29 | chr5_ [symbol] |
| Abx_Brs_1v4_07 | chr13_ [symbol] | Abx_Brs_1v7_20 | chr24_ [symbol] | Abx_Brs_1v4_40 | chr5_ [symbol] |
| Abx_Brs_1v5_ [symbol] | chr13_ [symbol] | Abx_Brs_1v3_15 | chr24_ [symbol] | Abx_Brs_1v [symbol] _37 | chr5_ [symbol] |
| Abx_Brs_1v7_05 | chr13_ [symbol] | Abx_Brs_1v2_21 | chr24_ [symbol] | Abx_Brs_1v3_30 | chr5_ [symbol] |
| Abx_Brs_1v7_09 | chr14_ [symbol] | Abx_Brs_1v7_21 | chr25_ [symbol] | Abx_Brs_1v5_22 | chr5_ [symbol] |
| Abx_Brs_1v3_ [symbol] | chr14_ [symbol] | Abx_Brs_1v2_22 | chr25_ [symbol] | Abx_Brs_1v2_33 | chr5_ [symbol] |
| Abx_Brs_1v7_10 | chr14_ [symbol] | Abx_Brs_1v [symbol] _24 | chr25_ [symbol] | Abx_Brs_1v5_23 | chr5_ [symbol] |
| Abx_Brs_1v4_09 | chr14_ [symbol] | Abx_Brs_1v6_25 | chr25_ [symbol] | Abx_Brs_1v7_ [symbol] | chr5_ [symbol] |
| Abx_Brs_1v0_12 | chr14_ [symbol] | Abx_Brs_1v3_17 | chr25_ [symbol] | Abx_Brs_1v [symbol] _32 | chr5_ [symbol] |
| Abx_Brs_1v7_08 | chr14_ [symbol] | Abx_Brs_1v2_22 | chr25_ [symbol] | Abx_Brs_1v4_41 | chr5_ [symbol] |
| Abx_Brs_1v4_ [symbol] | chr14_ [symbol] | Abx_Brs_1v3_18 | chr25_ [symbol] | Abx_Brs_1v6_38 | chr5_ [symbol] |
| Abx_Brs_1v3_07 | chr14_ [symbol] | Abx_Brs_1v6_ [symbol] | chr25_ [symbol] | Abx_Brs_1v2_35 | chr5_ [symbol] |
| Abx_Brs_1v2_04 | chr14_ [symbol] | Abx_Brs_1v7_23 | chr26_ [symbol] | Abx_Brs_1v3_33 | chr6_ [symbol] |
| Abx_Brs_1v5_09 | chr17_ [symbol] | Abx_Brs_1v [symbol] _27 | chr26_ [symbol] | Abx_Brs_1v7_32 | chr6_ [symbol] |
| Abx_Brs_1v7_11 | chr17_ [symbol] | Abx_Brs_1v4_22 | chr26_ [symbol] | Abx_Brs_1v6_39 | chr7_ [symbol] |
| Abx_Brs_1v2_ [symbol] | chr17_ [symbol] | Abx_Brs_1v3_19 | chr26_ [symbol] | Abx_Brs_1v2_ [symbol] | chr7_ [symbol] |
| Abx_Brs_1v2_ [symbol] | chr17_ [symbol] | Abx_Brs_1v4_23 | chr27_ [symbol] | Abx_Brs_1v5_24 | chr7_ [symbol] |
| Abx_Brs_1v4_10 | chr17_ [symbol] | Abx_Brs_1v6_28 | chr27_ [symbol] | Abx_Brs_1v [symbol] _40 | chr7_ [symbol] |
| Abx_Brs_1v7_12 | chr17_ [symbol] | Abx_Brs_1v2_23 | chr27_ [symbol] | Abx_Brs_1v5_25 | chr7_ [symbol] |
| Abx_Brs_1v4_11 | chr17_ [symbol] | Abx_Brs_1v2_24 | chr27_ [symbol] | Abx_Brs_1v4_42 | chr7_ [symbol] |
| Abx_Brs_1v7_13 | chr18_ [symbol] | Abx_Brs_1v6_29 | chr27_ [symbol] | Abx_Brs_1v2_37 | chr7_ [symbol] |
| Abx_Brs_1v6_13 | chr18_ [symbol] | Abx_Brs_1v4_24 | chr27_ [symbol] | Abx_Brs_1v6_41 | chr7_ [symbol] |
| Abx_Brs_1v2_07 | chr18_ [symbol] | Abx_Brs_1v6_30 | chr27_ [symbol] | Abx_Brs_1v4_43 | chr7_ [symbol] |
| Abx_Brs_1v5_10 | chr18_ [symbol] | Abx_Brs_1v2_ [symbol] | chr27_ [symbol] | Abx_Brs_1v4_44 | chr7_ [symbol] |
| Abx_Brs_1v2_ [symbol] | chr18_ [symbol] | Abx_Brs_1v2_28 | chr27_ [symbol] | Abx_Brs_1v4_48 | chr8_ [symbol] |
| Abx_Brs_1v [symbol] _14 | chr19_ [symbol] | Abx_Brs_1v2_27 | chr27_ [symbol] | Abx_Brs_1v4_ [symbol] | chr8_ [symbol] |
| Abx_Brs_1v4_12 | chr19_ [symbol] | Abx_Brs_1v7_24 | chr27_ [symbol] | Abx_Brs_1v7_33 | chr8_ [symbol] |
| Abx_Brs_1v5_11 | chr19_ [symbol] | Abx_Brs_1v [symbol] _31 | chr28_ [symbol] | Abx_Brs_1v7_34 | chr9_ [symbol] |
| Abx_Brs_1v2_10 | chr19_ [symbol] | Abx_Brs_1v5_17 | chr28_ [symbol] | Abx_Brs_1v7_35 | chrW_ [symbol] |
| Abx_Brs_1v5_12 | chr19_ [symbol] | Abx_Brs_1v4_25 | chr28_ [symbol] | Abx_Brs_1v5_26 | chrW_ [symbol] |
| Abx_Brs_1v2_09 | chr19_ [symbol] | Abx_Brs_1v7_25 | chr28_ [symbol] | Abx_Brs_1v4_47 | chrW_ [symbol] |
| Abx_Brs_1v [symbol] _14 | chr19_ [symbol] | Abx_Brs_1v7_26 | chr28_ [symbol] | Abx_Brs_1v4_49 | chrZ_ [symbol] |
| Abx_Brs_1v [symbol] _11 | chr2_ [symbol] | Abx_Brs_1v4_28 | chr28_ [symbol] | Abx_Brs_1v2_38 | chrZ_ [symbol] |
| Abx_Brs_1v [symbol] _15 | chr2_ [symbol] | Abx_Brs_1v4_27 | chr28_ [symbol] | Abx_Brs_1v3_34 | chrZ_ [symbol] |
| Abx_Brs_1v4_13 | chr2_ [symbol] | Abx_Brs_1v2_28 | chr3_ [symbol] | Abx_Brs_1v4_48 | chrZ_ [symbol] |
| Abx_Brs_1v6_16 | chr2_ [symbol] | Abx_Brs_1v4_29 | chr3_ [symbol] | | |
| Abx_Brs_1v2_12 | chr2_ [symbol] | Abx_Brs_1v2_29 | chr3_ [symbol] | | |

[symbol]

indicates data missing or illegible when filed

TABLE 7
Genome sites of the CpG cytosine residues differentially methylated in blood extracted
DNA from the control group compared to each individual antibiotic treatment group.

| | Chromosome_{—} | | Chromosome_{—} | | Chromosome_{—} |
|---|---|---|---|---|---|
| Internal ID | Methylated C | Internal ID | Methylated C | Internal ID | Methylated C |
| Abx_Bld_1v3_03 | chr1_105181389 | Abx_Bld_1v4_07 | chr19_4086429 | Abx_Bld_1v4_19 | chr3_16322261 |
| Abx_Bld_1v3_04 | chr1_112363445 | Abx_Bld_1v4_08 | chr19_5491370 | Abx_Bld_1v4_20 | chr3_17158125 |
| Abx_Bld_1v2_01 | chr1_112396072 | Abx_Bld_1v6_12 | chr19_5536508 | Abx_Bld_1v4_21 | chr3_25893764 |
| Abx_Bld_1v6_03 | chr1_128573573 | Abx_Bld_1v5_10 | chr19_8144962 | Abx_Bld_1v7_21 | chr3_3150548 |
| Abx_Bld_1v7_02 | chr1_144303500 | Abx_Bld_1v2_16 | chr19_8989012 | Abx_Bld_1v7_22 | chr3_3831201 |
| Abx_Bld_1v3_05 | chr1_154756172 | Abx_Bld_1v6_14 | chr2_115635929 | Abx_Bld_1v4_22 | chr3_66735580 |
| Abx_Bld_1v5_01 | chr1_156676 | Abx_Bld_1v5_13 | chr2_129424345 | Abx_Bld_1v6_18 | chr3_76106431 |
| Abx_Bld_1v3_06 | chr1_178417326 | Abx_Bld_1v3_21 | chr2_136077087 | Abx_Bld_1v7_23 | chr30_21732 |
| Abx_Bld_1v6_04 | chr1_194856444 | Abx_Bld_1v3_20 | chr2_18062865 | Abx_Bld_1v5_26 | chr4_11856003 |
| Abx_Bld_1v6_01 | chr1_1962010 | Abx_Bld_1v5_11 | chr2_22523723 | Abx_Bld_1v2_30 | chr4_43673227 |
| Abx_Bld_1v3_01 | chr1_50122971 | Abx_Bld_1v6_13 | chr2_315910 | Abx_Bld_1v3_31 | chr4_4578012 |
| Abx_Bld_1v6_02 | chr1_51169599 | Abx_Bld_1v5_12 | chr2_76061542 | Abx_Bld_1v3_32 | chr4_47166665 |
| Abx_Bld_1v7_01 | chr1_67153637 | Abx_Bld_1v6_15 | chr20_10090542 | Abx_Bld_1v6_20 | chr4_5380207 |
| Abx_Bld_1v3_02 | chr1_98606031 | Abx_Bld_1v7_11 | chr20_10096111 | Abx_Bld_1v3_33 | chr4_68799893 |
| Abx_Bld_1v5_04 | chr10_18811046 | Abx_Bld_1v3_23 | chr20_10100558 | Abx_Bld_1v5_27 | chr4_69408805 |
| Abx_Bld_1v3_08 | chr10_19473861 | Abx_Bld_1v5_16 | chr20_10870977 | Abx_Bld_1v3_34 | chr4_72510975 |
| Abx_Bld_1v2_02 | chr10_20081379 | Abx_Bld_1v4_09 | chr20_12980419 | Abx_Bld_1v4_23 | chr5_15848298 |
| Abx_Bld_1v4_01 | chr10_20160145 | Abx_Bld_1v5_15 | chr20_4046143 | Abx_Bld_1v2_32 | chr5_16029341 |
| Abx_Bld_1v5_03 | chr10_2654646 | Abx_Bld_1v3_22 | chr20_4835413 | Abx_Bld_1v6_22 | chr5_16988682 |
| Abx_Bld_1v5_02 | chr10_845224 | Abx_Bld_1v2_17 | chr20_488444 | Abx_Bld_1v2_33 | chr5_24963086 |
| Abx_Bld_1v3_07 | chr10_8640927 | Abx_Bld_1v5_14 | chr20_902666 | Abx_Bld_1v2_34 | chr5_26187561 |
| Abx_Bld_1v4_02 | chr11_1547400 | Abx_Bld_1v5_17 | chr21_1123800 | Abx_Bld_1v7_24 | chr5_27550747 |
| Abx_Bld_1v6_05 | chr11_17628332 | Abx_Bld_1v2_18 | chr21_2857891 | Abx_Bld_1v4_24 | chr5_30587252 |
| Abx_Bld_1v5_05 | chr11_17901197 | Abx_Bld_1v2_19 | chr21_3932464 | Abx_Bld_1v2_35 | chr5_32283491 |
| Abx_Bld_1v3_10 | chr11_19107727 | Abx_Bld_1v7_12 | chr22_2486087 | Abx_Bld_1v6_21 | chr5_370898 |
| Abx_Bld_1v3_09 | chr11_8438570 | Abx_Bld_1v4_10 | chr23_1877366 | Abx_Bld_1v7_25 | chr5_37861456 |
| Abx_Bld_1v3_11 | chr12_1097339 | Abx_Bld_1v3_24 | chr23_191817 | Abx_Bld_1v2_31 | chr5_399337 |
| Abx_Bld_1v4_03 | chr12_1126979 | Abx_Bld_1v7_13 | chr23_2040288 | Abx_Bld_1v3_35 | chr5_54483355 |
| Abx_Bld_1v3_12 | chr12_1306284 | Abx_Bld_1v5_18 | chr23_4262739 | Abx_Bld_1v6_23 | chr5_58816074 |
| Abx_Bld_1v2_05 | chr12_13166753 | Abx_Bld_1v4_11 | chr23_4632788 | Abx_Bld_1v2_36 | chr6_10306539 |
| Abx_Bld_1v2_03 | chr12_1328470 | Abx_Bld_1v6_16 | chr23_4958640 | Abx_Bld_1v7_27 | chr6_22248119 |
| Abx_Bld_1v7_03 | chr12_2397978 | Abx_Bld_1v2_20 | chr23_5304481 | Abx_Bld_1v7_28 | chr6_22301186 |
| Abx_Bld_1v2_04 | chr12_2418533 | Abx_Bld_1v6_17 | chr23_5357470 | Abx_Bld_1v5_28 | chr6_22875585 |
| Abx_Bld_1v7_04 | chr12_3220416 | Abx_Bld_1v4_12 | chr23_5497714 | Abx_Bld_1v3_36 | chr6_34299915 |
| Abx_Bld_1v6_06 | chr12_9058585 | Abx_Bld_1v2_21 | chr23_5507020 | Abx_Bld_1v5_29 | chr6_34299915 |
| Abx_Bld_1v3_14 | chr13_10139895 | Abx_Bld_1v3_25 | chr24_1676702 | Abx_Bld_1v7_29 | chr6_34911376 |
| Abx_Bld_1v2_06 | chr13_1032633 | Abx_Bld_1v7_14 | chr24_2238427 | Abx_Bld_1v7_26 | chr6_9225335 |
| Abx_Bld_1v5_07 | chr13_10441903 | Abx_Bld_1v2_22 | chr24_287622 | Abx_Bld_1v6_24 | chr7_22164945 |
| Abx_Bld_1v5_08 | chr13_12948949 | Abx_Bld_1v3_26 | chr24_5290703 | Abx_Bld_1v7_30 | chr7_22176711 |
| Abx_Bld_1v4_06 | chr13_13347784 | Abx_Bld_1v5_19 | chr24_5373054 | Abx_Bld_1v2_37 | chr7_22480250 |
| Abx_Bld_1v7_05 | chr13_15703345 | Abx_Bld_1v7_15 | chr25_1710811 | Abx_Bld_1v6_26 | chr8_14105687 |
| Abx_Bld_1v3_13 | chr13_1921148 | Abx_Bld_1v5_20 | chr25_1805702 | Abx_Bld_1v2_39 | chr8_21102815 |
| Abx_Bld_1v4_04 | chr13_4682096 | Abx_Bld_1v5_21 | chr25_1823943 | Abx_Bld_1v7_31 | chr8_23866489 |
| Abx_Bld_1v5_06 | chr13_9178753 | Abx_Bld_1v3_27 | chr25_2397267 | Abx_Bld_1v6_27 | chr8_27355783 |
| Abx_Bld_1v4_05 | chr13_9208825 | Abx_Bld_1v5_22 | chr25_2485917 | Abx_Bld_1v2_40 | chr8_29884082 |
| Abx_Bld_1v3_15 | chr14_12747204 | Abx_Bld_1v2_23 | chr25_2529119 | Abx_Bld_1v2_38 | chr8_5350190 |
| Abx_Bld_1v2_08 | chr14_13821711 | Abx_Bld_1v7_17 | chr26_1322363 | Abx_Bld_1v6_25 | chr8_9903290 |
| Abx_Bld_1v2_09 | chr14_14041698 | Abx_Bld_1v3_28 | chr26_3762706 | Abx_Bld_1v2_41 | chr9_14909691 |
| Abx_Bld_1v2_07 | chr14_2406601 | Abx_Bld_1v5_23 | chr26_404051 | Abx_Bld_1v6_28 | chr9_15840183 |
| Abx_Bld_1v6_07 | chr14_4945378 | Abx_Bld_1v2_25 | chr26_4164386 | Abx_Bld_1v6_29 | chr9_23157197 |
| Abx_Bld_1v3_16 | chr15_10852865 | Abx_Bld_1v2_26 | chr26_4812686 | Abx_Bld_1v5_31 | chr9_23470216 |
| Abx_Bld_1v3_17 | chr15_11248102 | Abx_Bld_1v4_13 | chr26_4812695 | Abx_Bld_1v4_27 | chr9_23509606 |
| Abx_Bld_1v6_10 | chr15_12394073 | Abx_Bld_1v2_24 | chr26_506540 | Abx_Bld_1v4_25 | chr9_4421109 |
| Abx_Bld_1v7_06 | chr15_7187787 | Abx_Bld_1v7_16 | chr26_627901 | Abx_Bld_1v3_37 | chr9_4853630 |
| Abx_Bld_1v6_09 | chr15_7376358 | Abx_Bld_1v2_27 | chr27_4283811 | Abx_Bld_1v5_30 | chr9_6531492 |
| Abx_Bld_1v2_10 | chr15_920285 | Abx_Bld_1v4_14 | chr27_445811 | Abx_Bld_1v4_26 | chr9_9246049 |
| Abx_Bld_1v6_08 | chr15_926910 | Abx_Bld_1v4_15 | chr27_4600438 | Abx_Bld_1v5_32 | chrZ_2038891 |
| Abx_Bld_1v6_11 | chr17_1959009 | Abx_Bld_1v4_16 | chr27_4813852 | Abx_Bld_1v3_38 | chrZ_21909666 |
| Abx_Bld_1v3_18 | chr17_5428705 | Abx_Bld_1v2_28 | chr27_4858045 | Abx_Bld_1v6_30 | chrZ_27238659 |
| Abx_Bld_1v3_19 | chr17_7760918 | Abx_Bld_1v4_17 | chr27_5078966 | Abx_Bld_1v7_32 | chrZ_27284938 |
| Abx_Bld_1v7_07 | chr17_9246565 | Abx_Bld_1v3_30 | chr28_1296205 | Abx_Bld_1v4_29 | chrZ_27627877 |
| Abx_Bld_1v5_09 | chr18_10428107 | Abx_Bld_1v7_18 | chr28_1399026 | Abx_Bld_1v7_33 | chrZ_39756286 |
| Abx_Bld_1v7_09 | chr18_10429145 | Abx_Bld_1v5_24 | chr28_1400149 | Abx_Bld_1v2_42 | chrZ_62115017 |
| Abx_Bld_1v7_10 | chr18_11039474 | Abx_Bld_1v7_19 | chr28_1411844 | Abx_Bld_1v6_31 | chrZ_68082819 |
| Abx_Bld_1v2_12 | chr18_7756868 | Abx_Bld_1v7_20 | chr28_3072184 | Abx_Bld_1v7_34 | chrZ_77245944 |
| Abx_Bld_1v7_08 | chr18_9116930 | Abx_Bld_1v4_18 | chr28_3600671 | Abx_Bld_1v6_32 | chrZ_78734434 |
| Abx_Bld_1v2_13 | chr18_9721568 | Abx_Bld_1v3_29 | chr28_863074 | Abx_Bld_1v6_33 | chrZ_81241584 |
| Abx_Bld_1v2_11 | chr18_988524 | Abx_Bld_1v2_29 | chr28_965371 | Abx_Bld_1v4_28 | chrZ_8328963 |
| Abx_Bld_1v2_14 | chr18_9889858 | Abx_Bld_1v5_25 | chr3_105678992 | | |
| Abx_Bld_1v2_15 | chr19_3856575 | Abx_Bld_1v6_19 | chr3_110316257 | | |

### Example 3

Impact of three classes of coccidiostats on the methylome of broiler chickens.

Modern broiler chickens are ubiquitously affected by protozoan parasites of the Eimeria spp. which results in an intestinal disease known as coccidiosis. Anticoccidial agents are therefore commonly utilized in broiler chicken management to prevent and manage coccidiosis.

A study is conducted to determine whether methylation of CpG sites in the genome of chicken breast tissue and blood is differentially affected by the administration of different types of anticoccidial agents and if the resultant epigenetic signatures can be used to create an analytic tool to measure anticoccidial usage in broilers from meat products.

A chicken trial was conducted from day of hatch to 42 days to represent the average slaughter age of broiler chickens in Europe. A total of 400 Ross308 male broilers are separated into 40 floor pens containing 10 birds/pen. Corn-soybean meal-based broiler diets are formulated to meet all nutrient requirements for starter (day 0-14), grower (day 14-28) and finisher (day 28-42) feeding phases. Treatments are created by adding treating the broilers with different anticoccidial agents (Table 7).

TABLE 7
Description of control and anticoccidial treatments in a 42 day broiler chicken trial.

| | | Pen | Dose and Mode of |
|---|---|---|---|
| Treatments | Anticoccidial Agent | Replicates | Administration |
| 1. Control | None | 10 | Not Applicable |
| 2. Ionophore | Monteban ®, narasin | 10 | 90 g/ton, in the feed |
| | (Elanco) | | |
| 3. Chemical Anticoccidial | Clinacox ®, diclazuril | 10 | 182 g/ton, in the feed |
| | (Elanco) | | |
| 4. Anti-Eimeria Vaccine | Coccivac B, live vaccine | 10 | Spray vaccination on Day 2 of |
| | (Merk Animal Health) | | broiler chick age |

Two animals per pen are sacrificed for sampling on days 28 and 42 for extraction of blood and various tissues including breast muscle tissue (Pectoralis major). A total of 160 samples are then utilized for DNA isolation with Invitrogen™ Purelink™ Genomic DNA Minikit (ThermoFisher Scientific, Waltham, MA, USA) and subsequent reduced bisulphite sequencing to compare methylation level of CpG sites in the genome.

Chicken samples for blood as well as for breast tissue are prepared with the Zymo RRBS protocol. The resulting libraries are sequenced and mapped to the Gallus gallus 5.0 genome using the tool bsmap 2.9. From the resulting methylation ratios all CpGs are kept, which had a non-zero coverage in all individual samples for a certain tissue.

To further analyze the ability to classify the samples based on the methylation data for treatments with different anticoccidocial agents, the R package randomForest is used and a random forest-based classification set up, using the 100,000 most variable CpGs in each tissue

First, the set of CpGs is split in chunks of 10,000 and each chunk is used to classify the samples into different categories, using a random forest. This is repeated 100 times. All CpGs having a positive score “MeanDecreaseAccuracy” in an individual classification are pooled and the top 10,000 CpGs, occurring most often, are kept. Then a second random forest is set up, using these selected 10,000 CpGs as features and a classification is performed. This is repeated 100 times and the average classification error, as well as the optimal classification error is evaluated.

The results show results indicate that treatment of broiler chickens with anticoccidial agents results in significant shifts to the CpG methylation profiles, which may therefore be used to differentiate anticoccidial—free from anticoccidial-treated poultry meat. The results also indicate that epigenetic signatures in the tissues of broiler chickens can be used to differentiate which anticoccidial type is utilized.

1. A method of determining if a test animal and/or a test animal from which a product is derived has been treated and/or is currently undergoing treatment with at least one antibiotic and/or veterinary chemical, the method comprising:
(a) determining a test methylation profile from the genomic material contained in a biological sample obtained from the test animal and/or the animal-derived product; and
(b) comparing the test methylation profile obtained from (a) with a reference methylation profile obtained from a control animal of the same biological taxon of the test animal, where the control animal was not treated and/or is not currently undergoing treatment with at least one antibiotic and/or veterinary chemical,
wherein a difference in the test methylation profile of (a) compared to the reference methylation profile from the control animal, is indicative of the test animal having been treated and/or is currently undergoing treatment with at least one antibiotic and/or veterinary chemical; and
wherein the test animal is a terrestrial animal; and
wherein the veterinary chemical is an anti-parasitic, an anti-viral, a feed additive, a water additive, a disinfectant, glutaraldehyde, and/or formalin.

2. The method according to claim 1, wherein the difference is hypomethylation or hypermethylation.

3. A method of determining if a test animal and/or a test animal from which a product is derived has been treated and/or is currently undergoing treatment with at least one antibiotic and/or veterinary chemical, the method comprising:
(a) determining the methylation status of at least one CpG site in genomic material contained in a biological sample obtained from the test animal,
(b) comparing the methylation status of the CpG site from (a) with that of a control animal of the same biological taxon as the test animal, which was not treated and/or is not currently undergoing treatment with at least one antibiotic and/or veterinary chemical,
wherein a difference in the test methylation status of (a) in the test animal compared to the CpG site in the control animal is indicative of the test animal having been treated and/or is currently undergoing treatment with at least one antibiotic and/or veterinary chemical; and
wherein the test animal is a terrestrial animal and
wherein the veterinary chemical is an anti-parasitic, an anti-viral, a feed additive, a water additive, a disinfectant, glutaraldehyde, and/or formalin.

4. The method according to claim 3, wherein the difference in methylation status is hypomethylation or hypermethylation of the CpG site in the test animal and the hypomethylation or hypermethylation of the CpG site is indicative of the test animal having been treated and/or is currently undergoing treatment with at least one antibiotic and/or veterinary chemical.

5. The method according to claim 3, wherein the CpG site is at least one CpG site selected from the Table below:

| No. | Chromosome | Position |
|---|---|---|
| 1 | 20 | 10783159 |
| 2 | 24 | 4184075 |
| 3 | 25 | 1910205 |
| 4 | 4 | 1712424 |
| 5 | 4 | 50270218 |
| 6 | 5 | 57738909 |
| 7 | 7 | 10956706 |
| 8 | 12 | 18699814 |
| 9 | 12 | 2415548 |
| 10 | 17 | 4082979 |
| 11 | 18 | 9932606 |
| 12 | 2 | 29649123 |
| 13 | 23 | 4069359 |
| 14 | 25 | 1445548 |
| 15 | 28 | 3467160 |
| 16 | 3 | 10490126 |
| 17 | 3 | 3328494 |
| 18 | 4 | 1627649 |
| 19 | 6 | 11522056 |
| 20 | 6 | 23106717 |
| 21 | 8 | 5481923 |
| 22 | 33 | 716102 |
| 23 | 22 | 4604122 |
| 24 | 22 | 4617202 |
| 25 | 23 | 502108 |
| 26 | 24 | 5683979 |
| 27 | 27 | 3015432 |
| 28 | 3 | 10479779 |
| 29 | 19 | 5380287 |
| 30 | 15 | 12638203 |
| 31 | 10 | 20073849 |
| 32 | 5 | 55805858 |

6. The method according to claim 1, wherein the animal-derived product is meat, processed meat, milk, collagen, feather, dairy products, blood and/or bone.

7. The method according to claim 1, wherein the animal derived product is from the terrestrial animal selected from the group consisting of chicken, lamb, cow, goat, sheep, pig, horse, donkey, turkey, duck, goose, quail, rabbit and mule.

8. The method according to claim 1, wherein the biological sample is selected from the group consisting of blood, brain, sperm, milk and any other tissue or sample that provides genomic DNA.

9. The method according to claim 1, wherein the antibiotic is selected from the group of classes consisting of amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim.

10. The method according to claim 1, wherein the feed additive is a coccidiostat or ionophore.

11. A method of determining if a test animal and/or a test animal from which a product is derived has been treated and/or is currently undergoing treatment with at least one antibiotic, and if so, determining the distinct class of antibiotics with which the test animal is being treated and/or is currently undergoing treatment, the method comprising:
(a) determining a test methylation profile from the genomic material contained in a biological sample obtained from the test animal and/or the animal-derived product sample; and
(b) comparing the test methylation profile obtained from (a) with one or more predetermined reference methylation profiles, wherein each of the predetermined reference methylation profiles is from a different animal of the same biological taxon as the test animal, and each of the different animals was treated with a different class of antibiotics,
wherein if the test methylation profile of (a) is significantly similar to one of the predetermined reference methylation profiles, the test animal is confirmed for being treated with the same class of antibiotics with which the animal with the predetermined reference profile is treated with and the test animal has been confirmed for having been treated and/or is currently undergoing treatment with the distinct class of antibiotics; and
wherein the test animal is a terrestrial animal.

12. The method according to claim 10, wherein the distinct classes of antibiotics are amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim.

13. A method of determining if a test animal and/or a test animal from which a product is derived has been treated and/or is currently undergoing treatment with at least one antibiotic, and if so, determining if the antibiotic is used as a growth promotant or as a therapeutant, the method comprising:
(a) determining a test methylation profile from the genomic material contained in a biological sample obtained from the test animal and/or the animal-derived product; and
(b) comparing the test methylation profile obtained from (a) with one or more predetermined reference methylation profiles, wherein each of the predetermined reference methylation profiles is from a different control animal of the same biological taxon as the test animal, and each of the different control animals was treated with the antibiotic as a growth promotant or therapeutant,
wherein if the test methylation profile of (a) is significantly similar to one of the predetermined reference methylation profiles, the test animal is confirmed for being treated with the antibiotic in the same way that the control animal with the similar predetermined reference profile is treated and the test animal has been confirmed for having been treated and/or is currently undergoing treatment with the antibiotic as a growth promotant or therapeutant; and
wherein the test animal is a terrestrial animal.

14. A method of determining if a test animal from which a product is derived underwent a withdrawal period of no treatment with at least one antibiotic and/or veterinary chemical prior to the product being obtained, the method comprising:
(a) determining a test methylation profile from the genomic material contained in a biological sample obtained from the animal-derived product; and
(b) comparing the test methylation profile obtained from (a) with at least two predetermined reference methylation profiles, wherein at least one of the predetermined reference methylation profiles is from a control animal that underwent a withdrawal period and at the other one of the predetermined reference methylation profiles is from a control animal of the same biological taxon as the test animal, that did not undergo a withdrawal period before the product was obtained,
wherein if the test methylation profile of (a) is significantly similar to the predetermined reference methylation profile, the test animal is confirmed for having undergone a withdrawal period or not; and
wherein the test animal is a terrestrial animal and
wherein the veterinary chemical is an anti-parasitic, an anti-viral, a feed additive, a water additive, a disinfectant, glutaraldehyde, and/or formalin.

15. A method of using DNA methylation profiling, comprising determining with DNA methylation profiling if a test animal and/or a test animal from which a product is derived has been treated and/or is currently undergoing treatment with at least one antibiotic and/or veterinary chemical, wherein the test animal is a terrestrial animal and the veterinary chemical is an anti-parasitic, an anti-viral, a feed additive, a water additive, a disinfectant, glutaraldehyde, and/or formalin.
